(19) United States
(12) Patent Application Publication
(10) Pub. No.: US 2005/0013973 A1
(43) Pub. Date: Jan. 20, 2005

(54) Implant

(76) Inventors: Paul Wilhelm Richter, Pretoria (ZA); Ugo Ripamonti, Constantia (ZA); Michael Edward Thomas, Pretoria (ZA); Gert Hendrik Jacobus Coetzee, Pretoria (ZA)

Correspondence Address: William S Frommer;Frommer Lawrence & Haug, 745 Fifth Avenue, New York, NY, 10151, US

(21) Appl. No.: 10/466,659

(22) PCT Filed: Jan. 14, 2002
(86) PCT No.: PCT/IB02/00080

(30) Foreign Application Priority Data
Jan. 19, 2001 (ZA) 2001/0575

(51) Int. Cl. B32B 1/00; A61F 2/28; B32B 1/10

(52) U.S. Cl. 428/158; 264/43; 623/23.5

(57) ABSTRACT

An artefact which is suitable for use as an implant is provided. The artefact includes a body having at least an outer surface layer of a calcium phosphate-based material. The outer surface layer has a surface area of at least 1,5m²/g. A plurality of micropores are provided in at least the outer surface layer of the body. The micropores have a maximum dimension of up to about 150 μm.

This invention relates to an implant. It relates in particular to an artefact which is suitable for use as an implant, and to a method of manufacture thereof.

According to a first aspect of the invention, there is provided an artefact which is suitable for use as an implant, the artefact including
a body having at least an outer surface layer of a calcium phosphate-based material, with the outer surface layer having a surface area of at least 1,5 m²/g; and
a plurality of micropores in at least the outer surface layer of the body, with the micropores having a maximum dimension of up to about 150 μm.

The Applicant believes that the artefact according to the invention will have. a sufficiently high degree of bioactivity so that it can be used as an implant, typically a bone implant. In particular, it is believed that the artefact will have enhanced bioactivity as compared to bone implants of calcium phosphate-based material but which do not have the high surface area and microporosity of the artefact according to the invention. Thus, the artefact will be osteoconductive, ie permitting bone growth onto its surface and into surface pores when it is in close proximity to viable bone. However, the artefact should preferably have sufficient bioactivity so that it is also osteoinductive, ie inducing bone growth onto its surface and into surface pores independently of the presence of viable bone near the implant, thereby rendering it particularly suitable for use as a bone implant. Furthermore, it is suitable for use as a soft tissue implant in a site where only soft tissue is in direct contact with the implant.

The Applicant has determined that an osteoinductive bone implant must have the combination of a high surface area, ie a surface area of at least 1,5 m³/g, and strong capillary action when immersed in liquid, such as water. The presence of micropores having a maximum dimension of 150 μm as hereinbefore discussed, promotes a strong capillary action. The necessary microporosity can be achieved by, for example, employing low temperature sintering of the calcium phosphate-based material during manufacture of the artefact, ie while sintering the artefact, limiting the maximum sintering temperature, Tₘₐₓ to ≦1050° C., with the micropores or interparticle pores thereby being formed.

The calcium phosphate-based material may, in particular, be a ceramic material. Thus, it may be hydroxyapatite. Hydroxyapatite is a sintered bioactive ceramic biomaterial.

In one embodiment of the invention, the entire body, ie not only the surface layer, may be of the calcium phosphate-based ceramic material having the microporosity, ie the plurality of micropores, as hereinbefore described. In other words, the entire body will then have a calcium phosphate-based ceramic structure.

However, in another embodiment of the invention, the body may comprise a core of dense material, and the outer surface layer, as hereinbefore described, covering the core. By ‘dense material’ is meant a material which has fewer of the micropores than the outer surface layer, ie has a lower concentration of the micropores than the outer surface layer, so that it has greater mechanical strength than the outer surface layer. The core may even be substantially devoid of any micropores. In one version of the invention, the core may be of a different material to that of the outer surface material. Thus, the core may then be a material which is chemically different to that of the surface layer. In another embodiment of the invention, the core may be of the same material as the outer surface layer save that it will, as hereinbefore set out, have a lower concentration of the micropores. In other words, the artefact will then have a mixed or graded structure comprising the relatively dense core and the outer surface layer as hereinbefore described, with both the core and the outer surface layer having the same chemical composition.

The surface area of the outer surface layer of the body may be from 2,0 m²/g to 2,5 m²/g, or even greater.

Macropores or macroporous spaces may be provided in the body. The macropores may be substantially spherical, and at least some may be interconnected. In particular, the macropores that are interconnected may be of spherical, intercoalesced form, ie adjacent macropores are coalesced together and thus not interconnected by elongate passageways. The macropores may be from 100 to 2000 microns in size, ie may have diameters of 100 to 2000 microns, preferably 400 to 800 microns.

All, or the majority of, the macropores may be of substantially the same size. The macropores may occupy from 20% to 80% of the total volume of that portion of the body in which they occur. For example, the macropores may occupy about 60% of the total volume of such portion of the body. The macropores may be randomly interspersed throughout said portion of the body. Thus, said portion of the body may have a network of interconnected coalesced rounded inner macroporous spaces.

However, it is also to be appreciated that most, and preferably all, of the macropores will be in communication with the outer surface of the artefact, eg by means of capillary passages. Thus, there will be few, if any, sealed or isolated macropores.

The micropores may have a maximum dimension of from sub-micron, eg about 50 nm, to 150 μm, typically from 1-10 μm. In one embodiment of the invention, the micropores, or some of the micropores, may be substantially spherical. However, in another embodiment of the invention, the majority, eg substantially all, of the micropores may be of irregular shape. The micropores may be randomly interspersed throughout the body. The micropores may be separate from one another, ie not connected together. The majority of the micropores may be of substantially the same size. The micropores may occupy 60% or less of the total volume of that portion of the body in which they occur, excluding the volume occupied by any macropores, ie the residual volume of that portion of the body after, the volume of any macropores has been excluded. Typically, the micropores may occupy about 40% of the residual body portion volume.

It will be appreciated that most, and preferably substantially all, of the micropores will be in communication with the outer surface of the artefact, eg by means of capillary passages. In other words, the calcium phosphate-based ceramic material will contain few, if any, sealed or isolated micropores.

The body may also, if desired, be provided with surface concavities, ie surface concavities in the outer surface layer. The surface concavities may have diameters of from 100 to 2000 microns, preferably 400 to 800 microns, and depths of 50 to 1000 microns, preferably 200 to 400 microns. The surface concavities may be hemispherical. The surface concavities may be interconnected with the macropores by being coalesced therewith.

According to a second aspect of the invention, there is provided a method of making an artefact which is suitable for use as an implant, the method including
mixing, at elevated temperature, calcium phosphate-based material in powder form with a thermoplastic binder, to produce a powder/binder mixture;
granulating the powder/binder mixture;
forming a green compact from the mixture; and
sintering the green compact, with the maximum temperature during the sintering being ≦1050° C., thereby to obtain an artefact comprising a sintered body having a surface area of at least 1,5 m²/g and a plurality of micropores interspersed throughout the body, with the micropores having a maximum dimension of up to about 150 μm.

The formation of the green compact may be effected by pressing, moulding or extruding the powder/binder mixture. When the formation of the green compact is by pressing, then the size of the granules of the powder/binder mixture is typically less than 500 μm. When the formation of the green compact is by moulding or extruding, then the size of the granules can either be less than 500 μm, or greater than 500 μm, eg up to several millimeters.

According to a third aspect of the invention, there is provided a method of making an artefact which is suitable for use as an implant, the method including
mixing, at elevated temperature, a mixture of a calcium, phosphate-based material in powder form and a powdered solid substance which is oxidizable into gaseous form, with a thermoplastic binder, to produce a powder/binder mixture;
granulating the powder/binder mixture;
forming a green compact from the mixture;
sintering the green compact at a temperature, T₁, and in a wet reducing or inert atmosphere, to obtain an artefact precursor;
cooling the precursor to a temperature, T₂, at which no further sintering takes place, while maintaining the wet reducing or inert atmosphere;
while maintaining the precursor at about T₂, exposing . it to an oxidizing environment, so as to oxidize at least some of the solid substance and render it into gaseous form, so that it is thereby substantially removed from the body, thereby to obtain an artefact comprising a sintered body having a surface area of at least 1,5 m²/g, with the spaces which were occupied by the solid. substance thus being micropores interspersed throughout the body and having a maximum dimension of up to about 150 μm.

The formation of the green compact may be effected by pressing, moulding or extruding the powder/binder mixture. When the formation of the green compact is by pressing, then the size of the granules of the powder/binder mixture is typically less than 500 μm. When the formation of the green compact is by moulding or extruding, then the size of the granules can either be less than 500 μm, or greater than 500 μm, eg up to several millimeters.

The powdered solid substance or oxidizable powder constituent is thus not oxidized during the sintering however, during the subsequent exposure-of the precursor to the oxidizing environment, at least some of this constituent is oxidized into gaseous form.

Sufficient of the powdered solid substance may be used so that the mass proportion of powdered solid substance to calcium phosphate based material in the powder/binder mixture is up to 3:2, and preferably about 1:3.

The calcium phosphate-based material or powder incorporated in the powder/binder mixture may have particle sizes from submicron, eg about 50 nm, to 100 μm. Preferably, the powdered calcium phosphate-based material has a narrow size distribution with a mean particle size of about 5 μm or less, eg about 1 μm. It is believed that this particle size distribution represents a balance between the powder being sufficiently fine to allow sintering yet being sufficiently coarse to permit achievement of high solids loading when mixed with the thermoplastic binder.

The powdered solid substance may be carbon. The carbon. particle size may be from submicron, eg about 50 nm, to 150 μm. Preferably, the carbon has a narrow size distribution, ie the particles are of substantially the same size, with a mean particle size of about 5 μm.

As set out hereinbefore, the calcium phosphate-based material may, in particular, be hydroxyapatite. The temperature at which hydroxyapatite powder sinters is dependent on its particle size. Typically, however, initial sintering occurs at about 950° C.-1000° C. Thus, T₁ is typically above 1100° C., eg is about 1200° C.

The atmosphere in which the sintering is effected may be a combination of a 5% hydrogen in nitrogen mixture, and steam.

The temperature, T₂, may be about 900° C.

The oxidizing atmosphere may be air.

It is believed that, in the method of the third aspect of the invention, high temperature sintering can be carried out without micropore closure or collapse. This is due the temporary presence of the carbon powder particles which inhibit or prevent micropore closure during the high temperature sintering, which allows sintering of adjacent calcium phosphate-based material to progress further. This in turn results in a stronger artefact.

The method of the third aspect of the invention has the further advantage that the shape and size of the micropores can be tailored, as desired. Thus, in one embodiment of the invention, the carbon particles may be smaller than the calcium phosphate-based material particles. The carbon particles will then, in the artefact precursor, occupy interstitial sites between hydroxyapatite particles. However, in another embodiment of the invention, the carbon particles may be of substantially the same size as the calcium phosphate-based material particles. The resultant micropores will then be of similar shape and size to the starting carbon particles, and have fundamentally different characteristics when compared to the case where the micropores are substantially smaller.

According to a fourth aspect of the invention, there is provided a method of making an artefact which is suitable for use as an implant, the method including
mixing, at elevated temperature, a mixture of a calcium phosphate-based material in powder form and a powdered solid substance which is oxidizable into gaseous form, with a thermoplastic binder, to produce a first powder/binder mixture;
granulating the first powder/binder mixture;
mixing, at elevated temperature, calcium phosphate-based material in-powder form with a thermoplastic binder, to produce a second powder/binder mixture containing no oxidizable powdered solid substance;
granulating the second powder/binder mixture;
forming the second powder/binder mixture into a core;
covering the core with an outer surface layer of the first powder/binder mixture, to obtain a green compact;
sintering the green compact at a temperature, T₁, and in a wet reducing or inert atmosphere, to obtain an artefact precursor;
cooling the precursor to a temperature, T₂₁, at which no further sintering takes place, while maintaining the wet reducing or inert atmosphere;
while maintaining the precursor at about T₂, exposing it to an oxidizing environment, so as to oxidize at least some of the solid substance and render it into gaseous form, so that it is thereby substantially removed from the body, thereby to obtain an artefact comprising a sintered body having an outer surface layer, with the outer surface layer having a surface area of at least 1,5 m²/g, with the spaces which were occupied by the solid substance thus being micropores interspersed throughout the surface layer and having a maximum dimension of up to about 150 μm.

As before, the formation of the green compact, ie the forming of the core and the covering thereof wit the outer surface layer, may be effected by pressing, moulding or extruding the powder/binder mixtures. When the formation of the green compact is by pressing, then the size of the granules of the first and second powder/binder mixtures is typically less than 500 μm. When the formation of the green compact is by moulding or extruding, then the size of the granules in the powder/binder mixtures can be less than 500 μm, or greater than 500 μm, eg up to several millimeters.

The powdered solid substance, T₁, T₃, the wet reducing or inert atmosphere, and the oxidizing atmosphere may thus be as hereinbefore described.

The core thus has few, if any, of the micropores.

Any suitable thermoplastic binder, such as a commercial polymeric binder used for extrusion or injection moulding of ceramic materials, may be used, provided it allows ambient temperature compaction of the granules to a strength adequate for further processing.

The temperature at which the mixing of the powders with the thermoplastic binder to produce the powder/binder mixtures takes place depends on the thermoplastic binder used, but is typically around 120° C.

The granulation of the powder/binder mixtures may be effected by crushing or milling the mixtures, and sieving them to the required granule or particle size.

The mixing of powder components may be effected by homogenizing the components in a ball mill for an extended period of time, eg for a period of several hours.

When it is desired to have macropores in the core and/or the outer surface layer of the artefact, fugitive phase particles which have sizes of 100 to 2000 microns and which are heat decomposable may be mixed with the relevant powder/binder mixture prior to the compaction of the green compact. Prior to sintering, the green compacts or bodies will then be heated to above the decomposition temperature of the fugitive phase particles, to form the macropores.

The fugitive phase particles may be stearic acid particles, which may be substantially spherical. The stearic acid particles will be selected such that they provide macropores or macroporous spaces of a desired size in the artefact. Thus, typically, stearic acid particles having a size range of 500 to 1000 microns may be used.

The relevant mixture is admixed with the fugitive phase particles in a desired mass ratio in order to provide a resultant artefact having a desired macropore volume. Thus, if a desired macropore volume of approximately 60% of the total artefact volume if desired, then the mass proportion of combined mixture to fugitive phase particles will be about 1:1,27 by mass.

To form the green compact or body, the mixture may be compacted at a pressure of about 20 MPa, moulded or extruded and machined, if necessary.

The temperature to which the green compacts or bodies are heated is dependent on the fugitive phase used. However, when stearic acid particles are used as the fugitive phase, the green compacts are typically heated to about 500° C., to allow melting and decomposition of the stearic acid, thereby forming in the green compacts or bodies, interconnected macropores produced by the decomposition of the stearic acid particles. When sintering in air without an oxidizable component, the sintering temperature and time is set or limited by the level of micropores required in the resultant implant. For example, to obtain a total microporosity level of 40% by volume in the resultant implant, sintering may be effected at about 1020° C. for one hour.

The invention will now be described by way of non-limiting example, with reference to the accompanying drawings which show simplified views of artefacts according to the invention.

### IN THE DRAWINGS

FIG. 1 shows a cross-sectional view of an artefact according to a first embodiment of the invention, and

FIG. 2 Shows a sectional view of an artefact according to a second embodiment of the invention.

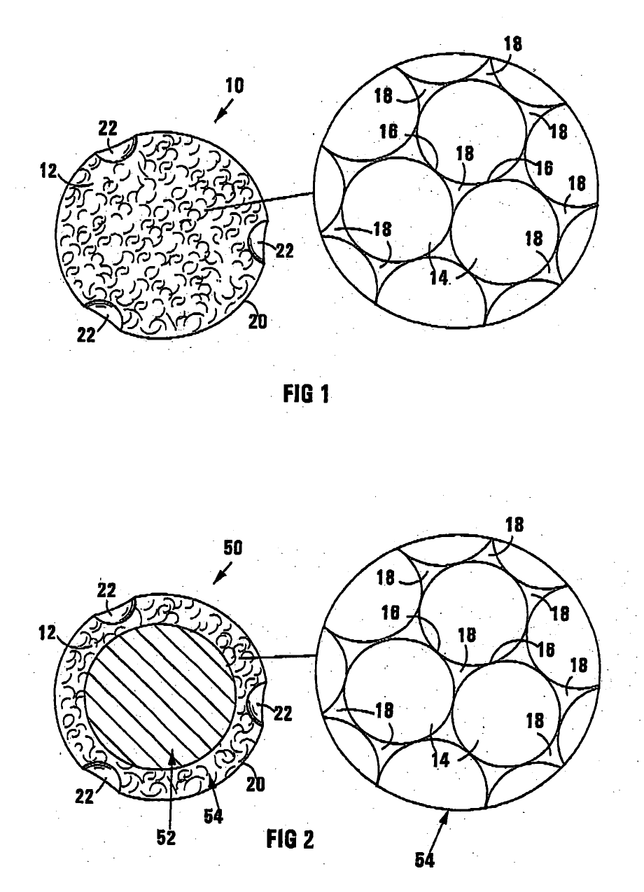

Referring to FIG. 1, reference numeral 10 generally indicates an artefact according to a first embodiment of the invention.

The artefact 10 includes a body 12 of hydroxyapatite. The body 12 comprises a plurality of particles 14 of hydroxyapatite, which are sintered, ie fused, together in zones 16 where the particles touch each other, so that irregular shaped micropores 18 are formed between the particles 14. The micropores 18 have a maximum dimension of 10 μm at most, and typically have a maximum dimension in the range of 1-10 μm. The micropores 18 are interspersed throughout the body 12.

The body 12 has an outer surface 20 in which are provided a plurality of surface concavities 22. The surface concavities 22 are hemispherical in cross-section, and may have dimensions of 400-800 μm, and depths of 200-400 μm. The concavities 22 are irregularly or randomly spaced in the outer surface 20.

The outer surface 20, which thus includes the surfaces of the concavities 22, has a surface area of at least 1,5 m²/g, and preferably 2,0-2,5 m²/g.

It is to be appreciated that a simplified view of the artefact is shown in FIG. 1. In practice, the zones 16 will not be as clearly demarcated as shown in FIG. 1. Instead, adjacent hydroxyapatite particles 14 will flow or merge into each other to greater or lesser extent, depending on degree of sinter of the adjacent particles. As a result, the micropores 18 will in practice not have the same shapes and sizes as indicated in the drawing; instead, their shapes and sizes will be dictated by the degree of sinter of adjacent particles. In other words, most, if not all, of the micropores 18 will be of different shape and/or size. Still further, the micropores 18 is will not normally, in practice, be arranged in a regular pattern as indicated in the drawing; instead, they will be randomly arranged depending on the degree of sinter of the particles. Thus, for example, a number of the particles 14 may be fully sintered and thus wholly integral with one another, with no micropores being defined between such particles.

Additionally, substantially none of the micropores 18 will be sealed or isolated, ie substantially all of the micropores 18 will be in communication with the outer surface 20 by means of capillary passages (not shown).

Referring now to FIG. 2, reference numeral 50 generally indicates an artefact according to another embodiment of the invention.

Parts of the artefact 50 which are the same or similar to those of the artefact 10 hereinbefore described with reference to FIG. 1, are indicated with the same reference numerals.

The body 12 of the artefact 50 comprises a core 52 of dense hydroxyapatite material, ie hydroxyapatite material that is devoid of any pores, particularly micropores 18. The core 52 is covered by an outer surface layer 54 of hydroxyapatite material having the particles 14 and the micropores 18. The surface layer 54 thus also has the outer surface 20 with the concavities 22.

The artefacts 10, 50 are suitable for use as bone implants having both osteoconductivity and osteoinductivity. The implants 10, 50 thus exhibit high surface area and strong capillary action when immersed in body fluid such as blood, by virtue of the high level of microporosity of the implant surface.

The artefacts 10, 50 can be manufactured in accordance with Examples 1 to 4 hereunder, with the artefact 10 being produced by the method of Examples 1 and 2, and the artefact 50 by the method of Examples 3 and 4.

### EXAMPLE 1

A green artefact is formed by compounding hydroxyapatite powder, having a narrow size distribution and a mean particle size of about 5 μm, with a commercial thermoplastic polymeric binder at a temperature of about 120° C., to produce a powder/polymer mixture. This mixture in crushed and sieved to a particle size smaller than 300 μm. In this fashion, a granular mixture is obtained.

Any commercial thermoplastic polymeric binder suitable for extrusion or injection moulding of ceramic materials, may be used, provided it allows ambient temperature compaction of the granules of the mixture to a strength adequate for further processing.

The mixture is pressed or compacted, in a suitable die or mould, at a pressure of 20 MPa, and machined if necessary. In this fashion green compacts are obtained. The die or mould will typically be provided with protrusions for forming the cavities 22 in the outer surfaces of the green compacts.

The green compacts are heated, in a furnace, to a temperature of 1050° C. for sintering of the hydroxyapatite powder. Due to the low sintering temperature or undersintering, adjacent particles merely sinter together where they abut, ie there is an absence of total fusion or merging of particles into one another. Irregular shaped micropores having a maximum size or dimension of 1-10 μm, are thus formed between particles.

The green artefact is sintered at a relatively low temperature of 1050° C. to obtain the artefact 10 of FIG. 1.

### EXAMPLE 2

A green artefact is formed by following the same general procedure as described in Example 1 except that 25% by mass of the hydroxyapatite powder is replaced by carbon powder, which is thus intimately admixed with the hydroxyapatite powder.

The green compact that is obtained is sintered at a relatively high temperature of 1200° C., under a slightly reducing atmosphere of the combination of 5% (by volume) hydrogen in nitrogen mixture, and steam. The body material is then allowed to cool to 900° C. in the furnace, with this temperature being too low for further sintering to take place. Air is admitted to the furnace at this temperature over an extended period of several hours. The air results in the carbon being oxidized and removed as a gas, thereby yielding the artefact 10 having the fine microporous structure and the high surface area as hereinbefore described.

Due to the higher sintering temperature in this Example as compared to the sintering temperature used in Example 1, sintering between adjacent particles progresses to a greater degree, resulting in a stronger artefact, as hereinbefore described.

Using the method of this Example, an artefact as illustrated in FIG. 1 is obtained when the carbon powder particles also have a narrow size distribution, and with their mean particle size an order of magnitude smaller than the hydroxyapatite particles. When the mean particle size of the carbon powder particles is similar to that of the hydroxyapatite particles, the micropores 18 will be of similar size and shape to the carbon powder particles.

### EXAMPLE 3

In order to produce the artefact 50, a hydroxyapatite/carbon granular mixture as described in Example 2, is made up (‘Component 1’). A standard hydroxyapatite granular mixture as described in Example 1 is also prepared (‘Component 2’). An amount of Component 1 is introduced into a pressing die and slightly compacted into disc form. An amount of Component 2 is then deposited on top of the slightly compacted disc of Component 1, while still in the die, levelled and thereafter slightly compacted. A third layer of Component 1 is then added to the stack in the die. The stack is then compacted under hydraulic pressure to yield a green artefact comprising standard hydroxyapatite powder (Component 2) sandwiched between two outer layers of carbon containing hydroxyapatite powder (Component 1).

This green artefact is then sintered at a high temperature of 1200° C. under a slightly reducing atmosphere of the combination of 5% (by volume) hydrogen in nitrogen mixture, and steam. The material is thereafter allowed to cool to 900° C. in the furnace, with this temperature being too low for further sintering to take place. Air is admitted into the furnace at this temperature over an extended period of several hours. The carbon in the outer layer of the artefact precursor in oxidized and removed as a gas. The resultant artefact comprises a relatively dense hydroxyapatite inner core 52 of high strength, with an outer layer 54 of microporous hydroxyapatite of high surface area and enhanced bioactivity as hereinbefore described.

### EXAMPLE 4

This example describes how an elongated artefact with cross-section similar to that of the compacted artefact 50 can be produced by means of extrusion. A hydroxyapatite/carbon powder mixture as described in Example 2 is compounded with the polymeric binder to produce an extrudable component (‘Feedstock 1’).

A hydroxyapatite powder as described in Example 1, is a compounded with the binder to produce a second extrudable component (‘Feedstock 2’). Feedstocks 1 and 2 are co-extruded at an elevated temperature appropriate for the particular binder used, to yield an inner rod-like core of Feedstock 2; covered by an outer sleeve-like layer of Feedstock 1. This green artefact is then sintered, cooled and subjected to air treatment as described in Example 3, to yield an artefact having a relatively dense inner core 52 of high strength and a microporous outer surface layer 54 having enhanced bioactivity as hereinbefore described. Concavities 22 may also be introduced on the outer surface of the extruded body by repeatedly indenting the surface of the extrudate as it emerges from the extrusion nozzle.

### EXAMPLES 5 to 8

Examples 1 to 4 were repeated, in Examples 5 to 8 respectively, using identical constituents, parameters, etc as in Examples 1 to 4, except for the following:
In Example 5 (which corresponds to Example 1), the hydroxyapatite powder had a mean particle size of about 1 μm
In Example 5, the irregular shaped micropores that were formed between the particles had a maximum size or dimension of less then 10 μm;
In Example 7 (which corresponds to Example 3), the amount of Component 1 that as introduced into the pressing die was slightly compacted into a cylindrical form. An amount of Component 2 was placed in a different die and slightly compacted to a disc form. The disc form manufactured of Component 2 was then placed in the cylinder form manufactured of Component 1, and the entire structure consolidated by compaction to a higher pressure of 20 MPa. The resulting green artefact then comprised a disc of standard hydroxyapatite powder (Component 2) enclosed by a ring of carbon containing hydroxyapatite powder (Component 1);
In Example 8 (which corresponds to Example 4), the hydroxyapatite/carbon powder mixture of Example 6 (which corresponds to Example 2) and which thus included the hydroxyapatite powder of Example 5 rather than that of Example 1, was used.

1. An artefact which is suitable for use as an implant, the artefact including
a body having at least an outer surface layer of a calcium phosphate-based material, with the outer surface layer having a surface area of at least 1,5 m²/g; and
a plurality of micropores in at least the outer surface layer of the body, with the micropores having a maximum dimension of up to about 150 μm.

2. An artefact according to claim 1, wherein the calcium phosphate-based material is hydroxyapatite.

3. An artefact according to claim 1, wherein the entire body is of the calcium phosphate-based ceramic material having the plurality of micropores

4. An artefact according to claim 1, wherein the body comprises a core of dense material, and the outer surface layer which covers the core.

5. An artefact according to claim 4, wherein the core is substantially devoid of any micropores.

6. An artefact according to claim 4, wherein the core is of a different material to that of the outer surface layer material.

7. An artefact according to claim 4, wherein the core is of the same material as the outer surface layer save that it has a lower concentration of the micropores

8. An artefact according to claim 1, wherein the surface area of the outer surface layer of the body is at least 2,0 m²/g.

9. An artefact according to claim 1, wherein macropores are provided in the body.

10. An artefact according to claim 9, wherein the macropores are substantially spherical, and at least some of them are interconnected, with the macropores that are interconnected being of spherical, intercoalesced form so that adjacent macropores are coalesced together.

11. An artefact according to claim 10, wherein the macropores are from 100 to 2000 microns in size.

12. An artefact according to claim 9, wherein the majority of the macropores are of substantially the same size, and/or wherein the macropores occupy from 20% to 80% of the total volume of that portion of the body in which they occur, and/or wherein the macropores are randomly interspersed throughout that portion of the body in which they occur.

13. An artefact according to claim 9, wherein substantially all of the macropores are in communication with the outer surface of the artefact by means of capillary passages.

14. An artefact according to claim 9, wherein the maximum dimension of the micropores is from sub-micron to 150 μm.

15. An artefact according to claim 14, wherein the majority of the micropores are substantially spherical.

16. An artefact according to claim 14, wherein the majority of the micropores are of irregular shape.

17. An artefact according to claim 9, wherein the micropores are randomly interspersed throughout the body; and/or wherein the micropores are separate from one another; and/or wherein the majority of the micropores are of substantially the same size; and/or wherein the micropores occupy 60% or less of the total volume of that portion of the body in which they occur, excluding the volume occupied by any macropores.

18. An artefact according to claim 1, wherein substantially all of the micropores are in communication with the outer surface of the artefact by means of capillary passages so that the calcium phosphate-based ceramic material contains substantially no sealed or isolated micropores.

19. An artefact according to claim 1, wherein hemispherical surface concavities are provided in the outer surface layer of the body, with the surface concavities having diameters of from 100 to 2000 microns and depths of 50 to 1000 microns.

20. A method of making an artefact which is suitable for use as an implant, the method including
mixing, at elevated temperature, calcium phosphate-based material in powder form with a thermoplastic binder, to produce a powder/binder mixture;
granulating the powder/binder mixture;
forming a green compact from the mixture; and
sintering the green compact, with the maximum temperature during the sintering being ≦1050° C., thereby to obtain an artefact comprising a sintered body having a surface area of at least 1,5 m²/g and a plurality of micropores interspersed throughout the body, with the micropores having a maximum dimension of up to about 150 μm.

21. A method of making an artefact which is suitable for use as an implant, the method including
mixing, at elevated temperature, a mixture of a calcium phosphate-based material in powder form and a powdered solid substance which is oxidizable into gaseous form, with a thermoplastic binder, to produce a powder/binder mixture;
granulating the powder/binder mixture;
forming a green compact from the mixture;
sintering the green compact at a temperature, T₁, and in a wet reducing or inert atmosphere, to obtain an artefact precursor;
cooling the precursor to a temperature, T₂, at which no further sintering takes place, while maintaining the wet reducing or inert atmosphere;
while maintaining the precursor at about T₂, exposing it to an oxidizing environment, so as to oxidize at least some of the solid substance and render it into gaseous form, so that it is thereby substantially removed from the body, thereby to obtain an artefact comprising a sintered body having a surface area of at least 1,5 m²/g, with the spaces which were occupied by the solid substance thus being micropores interspersed throughout thc body and having a maximum dimension of up to about 150 μm.

22. A method according to claim 21, wherein the powdered calcium phosphate-based material is hydroxyapatite, with the hydroxyapatite particles having a narrow size distribution and a mean particle size of about 1 μm.

23. A method according to claim 22, wherein the powdered solid substance is carbon, with the carbon particles having a narrow size distribution and a mean particle size of about 5 μm.

24. A method according to claim 23, wherein the formation of the green compact is effected by pressing, moulding or extruding the mixture; and/or wherein the temperature, T₁, is above 1100° C.; and/or wherein the atmosphere in which the sintering is effected is a combination of a 5% hydrogen in nitrogen mixture, and steam; and/or wherein the temperature, T₂, is about 900° C.; and/or wherein the oxidizing environment is air; and/or wherein the mass proportion of carbon to hydroxyapatite in the powder/binder mixture is about 1:3.

25. A method according to claim 23, wherein the carbon particles are smaller than the hydroxyapatite particles so that the carbon particles in the artefact precursor occupy interstitial sites between hydroxyapatite particles.

26. A method according to claim 23, wherein the carbon particles are of substantially the same size as the hydroxyapatite particles so that the resultant micropores are of similar shape and size to the starting carbon particles.

27. A method of making an artefact which is suitable for use as an implant, the method including
mixing, at elevated temperature, a mixture of a calcium phosphate-based material in powder form and a powdered solid substance which is oxidizable into gaseous form, with a thermoplastic binder, to produce a first powder/binder mixture;
granulating the first powder/binder mixture;
mixing, at elevated temperature, calcium phosphate-based material in powder form with a thermoplastic binder, to produce a second powder/binder mixture containing no oxidizable powdered solid substance;
granulating the second powder/binder mixture;
forming the second powder/binder mixture into a core;
covering the core with an outer surface layer of the first powder/binder mixture, to obtain a green compact;
sintering the green compact at a temperature, T₁, and in a wet reducing or inert atmosphere, to obtain an artefact precursor;
cooling the precursor to a temperature, T₂, at which no further sintering takes place, while maintaining the wet reducing or inert atmosphere;
while maintaining the precursor at about T₂, exposing it to an oxidizing environment, so as to oxidize at least some of the solid substance and render it into gaseous form, so that it is thereby substantially removed from the body, thereby to obtain an artefact comprising a sintered body having an outer surface layer, with the outer surface layer having a surface area of at least 1,5 m²/g, with the spaces which were occupied by the solid substance thus being micropores interspersed throughout the surface layer and having a maximum dimension of up to about 150 μnm.

28. A method according to claim 27, wherein the powdered calcium phosphate-based material is hydroxyapatite, with the hydroxyapatite particles having a narrow size distribution and a mean particle size of about 1 μm.

29. A method according to claim 28, wherein the powdered solid substance is carbon, with the carbon particles having a narrow size distribution and a mean particle size of about 5 μm.

30. A method according to claim 29, wherein the formation of the core and the covering thereof with the outer surface layer is effected by pressing, moulding or extruding the powder/binder mixture; and/or wherein the temperature, T₁, is above 1100° C.; and/or wherein the atmosphere in which the sintering is effected is a combination of a 5% hydrogen in nitrogen mixture, and steam; and/or wherein the temperature, T₂, is about 900° C.; and/or wherein the oxidizing environment is air; and/or wherein the mass proportion of carbon to hydroxyapatite in the powder/binder mixture is about 1:3.

31. A method according to claim 27, wherein the granulation of the powder/binder mixtures is effected by crushing or milling the mixtures, and sieving them to the required granule or particle size.

32. A method according to claim 27, wherein the mixing of the powder components is effected by homogenizing the components in a ball mill for an extended period of time.

33. A method according to claim 27, wherein fugitive phase particles which have sizes of 100 to 2000 microns and which are heat decomposable, are mixed with the first powder/binder mixture and/or with the second powder/binder mixture, with the green compact, prior to sintering, being heated to above the decomposition temperature of the fugitive phase particles, thereby to form macropores.

34. A method according to claim 33, wherein the fugitive phase particles are stearic acid particles which are substantially spherical, with the stearic acid particles having a size range of 500 to 1000 microns.

35-36. (canceled)
